(19) United States
(12) Patent Application Publication
(10) Pub. No.: US 2010/0016909 A1
(43) Pub. Date: Jan. 21, 2010

(54) IMPLANTABLE CARDIAC DEVICE FEEDTHRU/HEADER ASSEMBLY

(75) Inventors: Wambui Gachiengo, Los Angeles, CA (US); Narendra Nayak, Santa Clarita, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/175,349

(22) Filed: Jul. 17, 2008

(51) Int. Cl. A61N 1/372

(52) U.S. Cl. 607/4

(57) ABSTRACT

In one embodiment, an ICD is provided which includes a case having a connector block and a conductor post integrally formed with the connector block and extending through a dielectric feedthrough extending through the case. A capacitor is located within the dielectric. In some embodiments, the conductor post is a straight conductor post extending from a side of the connector block facing the feedthrough directly toward the feedthrough. The conductor post and the connector block may be formed of the same material, such as titanium. In some embodiments, a plurality of straight conductor posts and connector blocks are integrally formed. In some embodiments, the dielectric may be a single matrix dielectric, such that each of the straight conductor posts extends through the single matrix dielectric. In other embodiments, each of the straight conductor posts extends through a separate dielectric portion.

### BACKGROUND

FIG. 1 illustrates an ICD or implantable cardiac device 10 in electrical communication with a patient's heart 12 by way of three leads, 20, 24, and 30, suitable for delivering multi-chamber stimulation and shock therapy. FIG. 2 shows a portion of a conventional feedthrough assembly 205 of the implantable cardiac device 10. The feedthrough assembly 205 has platinum iridium wires 225 which are welded to titanium connector blocks 235. The platinum wires 225 extend through case 40 of the implantable cardiac device 10 via feedthrough ceramic 215. The platinum iridium wires 225 are bent to position the connector blocks 235. This requires manipulation of the wires 225 to form them, and multiple weld/brazing joints (not shown) to secure the wires 225 to their respective ceramic substrates 215 and to secure the flange portions 245 of the substrate 215 to the case 40.

The manufacturing process is labor intensive. It often requires rework of wires 225, as wires 225 can move in the process of molding or casting of the header (epoxy header not shown). Moreover, the platinum iridium is very expensive. Thus, this results in an expensive feedthrough assembly 205 due to the use of platinum wires 225, and due to a cumbersome and variable process required to form and insert the shaped wires 225.

Furthermore, sometimes an error in wire 225 formation can result in the high voltage wires 225 getting too close to the case 40. Since typical high voltage defibrillation therapy is about 800V, positioning the wires 225 too close to the case 40 could cause shorting during delivery of defibrillation therapy, leading to catastrophic failure.

What is needed is a significant reduction in costs without sacrificing reliability. In addition, what is needed is a way to reduce manufacturing complexity and at the same time increase the reliability of the header assembly.

### SUMMARY

In one implementation, an implantable cardiac device is provided which includes a case having a connector block and a conductor post integrally formed with the conductor post extending through a dielectric feedthrough which extends through the case. A capacitor is located within the dielectric.

In some embodiments, the conductor post is a straight conductor post extending from a side of the connector block facing the feedthrough directly toward the feedthrough. The conductor post and the connector block may be formed of the same material, such as titanium. Other suitable materials include MP35N, stainless steel, palladium, platinum-iridium, and the like.

In some embodiments, a plurality of straight conductor posts and connector blocks are integrally formed. In some embodiments, the dielectric may be a single matrix dielectric, such that each of the straight conductor posts extends through the single matrix dielectric. In other embodiments, each of the straight conductor posts extends through a separate dielectric portion.

### BRIEF DESCRIPTION OF THE DRAWINGS

Further features and advantages of the invention may be more readily understood by reference to the following description taken in conjunction with the accompanying drawings, in which:

FIG. 1 illustrates a prior art implantable cardiac device in electrical communication with a patient's heart.

FIG. 2 shows a portion of a conventional feedthrough header assembly of the implantable cardiac device.

FIG. 3 illustrates a simplified block diagram of a prior art stimulation device.

FIG. 4 shows a portion of a feedthrough header assembly of an implantable cardiac device in accordance with one embodiment of the present invention.

FIG. 5 shows a perspective view of a molded header.

FIG. 6 shows a perspective view of an implantable cardiac device with an attached header assembly.

FIG. 7 shows a perspective view of a possible embodiment of the feedthrough assembly having individual dielectric rings for each of the conductor posts.

### DESCRIPTION

The following description includes the best mode presently contemplated for practicing the described implementations. This description is not to be taken in a limiting sense, but rather is made merely for the purpose of describing the general principles of the implementations. The scope of the described implementations should be ascertained with reference to the issued claims. In the description that follows, like numerals or reference designators will be used to reference like parts or elements throughout.

### Overview of Implantable Cardiac Stimulation Device

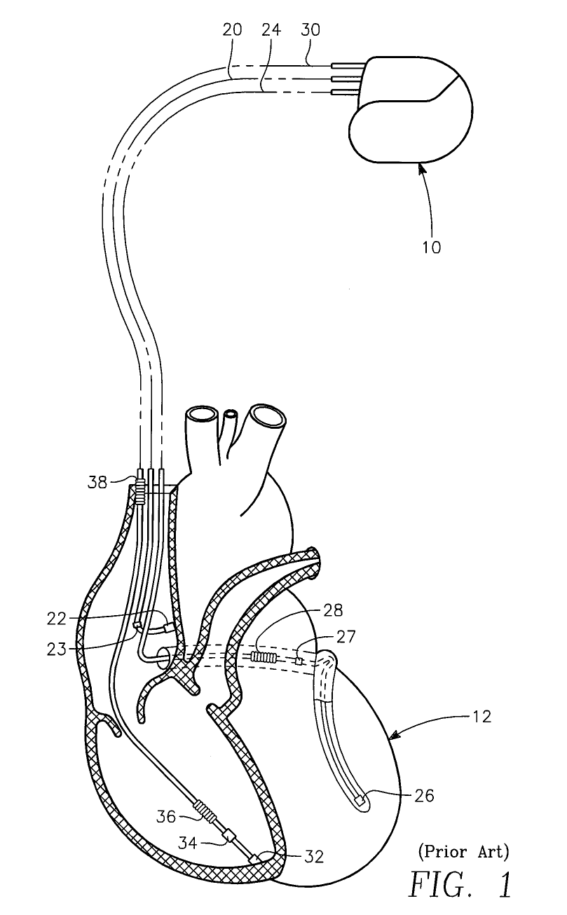

FIG. 1 illustrates an implantable cardiac stimulation device 10 in electrical communication with a patient's heart 12 by way of three leads, 20, 24 and 30, suitable for delivering multi-chamber stimulation and shock therapy. To sense atrial cardiac signals and to provide right atrial chamber stimulation therapy, the stimulation device 10 is coupled to an implantable right atrial lead 20 having at least an atrial tip electrode 22, which typically is implanted in the patient's right atrial appendage, and an atrial ring electrode 23. To sense left atrial and ventricular cardiac signals and to provide left chamber pacing therapy, the stimulation device 10 is coupled to a “coronary sinus” lead 24 designed for placement in the “coronary sinus region” via the coronary sinus or for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, in some embodiments, an exemplary coronary sinus lead 24 is designed to receive atrial and ventricular cardiac signals and to deliver left ventricular pacing therapy using at least a left ventricular tip electrode 26, left atrial pacing therapy using at least a left atrial tip electrode 27, and shocking therapy using at least a left atrial coil electrode 28.

The stimulation device 10 is also shown in electrical communication with the patient's heart 12 by way of an implantable right ventricular lead 30 having, in this embodiment, a right ventricular tip electrode 32, a right ventricular ring electrode 34, a right ventricular (RV) coil electrode 36, and a superior vena cava (SVC) coil electrode 38. Typically, the right ventricular lead 30 is transvenously inserted into the heart 12 so as to place the right ventricular tip electrode 32 in the right ventricular apex so that the right ventricular coil electrode 36 will be positioned in the right ventricle and the SVC coil electrode 38 will be positioned in the superior vena cava. Accordingly, the right ventricular lead 30 is capable of receiving cardiac signals, and delivering stimulation in the form of pacing and shock therapy to the right ventricle.

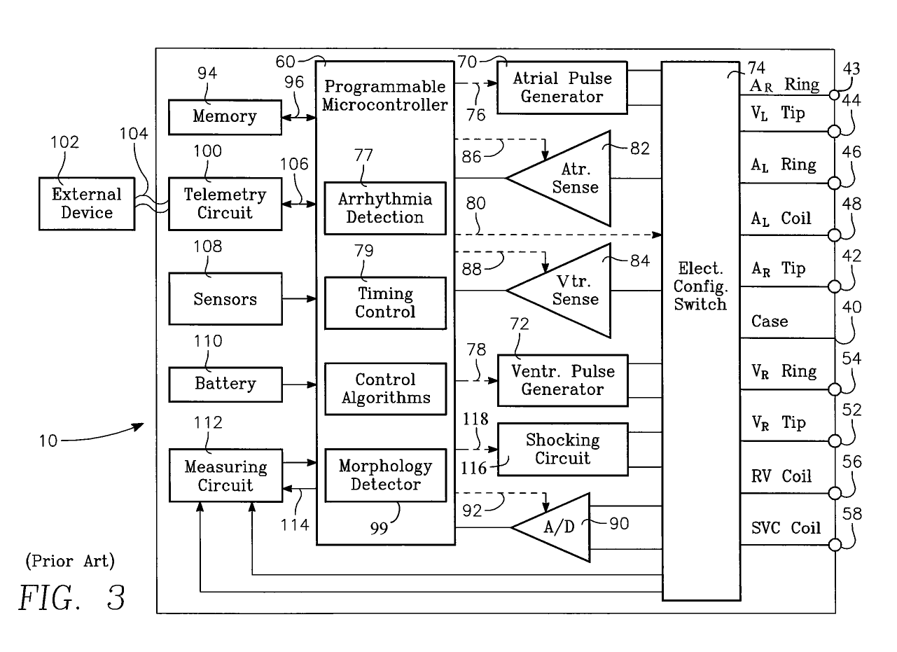

FIG. 3 illustrates a simplified block diagram of the stimulation device 10. The stimulation device 10 is capable of treating both fast and slow arrhythmias with stimulation therapy, including cardioversion, defibrillation, and pacing stimulation. While a particular stimulation device 10 is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation.

The stimulation device 10 includes a case 40. The case 40 for the stimulation device 10, shown schematically in FIG. 3, is often referred to as the “housing”, “can”, or “case electrode” and may be programmably selected to act as the return electrode for all “unipolar” modes. The case 40 may further be used as a return electrode individually or in combination with one or more of the coil electrodes, 28, 36 and 38, for shocking purposes. The case 40 further includes a connector (not shown) having a plurality of terminals, 42, 43, 44, 46, 48, 52, 54, 56, and 58 (shown schematically and, for convenience, the names of the electrodes to which they are connected are shown next to the terminals). As such, to achieve right atrial sensing and pacing, the connector includes at least a right atrial tip terminal (A_{R} TIP) 42 adapted for connection to the atrial tip electrode 22 and a right atrial ring (A_{R} RING) terminal 43 adapted for connection to right atrial ring electrode 23. To achieve left chamber sensing, pacing and shocking, the connector includes at least a left ventricular tip terminal (V_{L} TIP) 44, a left atrial ring terminal (A_{L} RING) 46, and a left atrial shocking terminal (A_{L} COIL) 48, which are adapted for connection to the left ventricular tip electrode 26, the left atrial tip electrode 27, and the left atrial coil electrode 28, respectively. To support right chamber sensing, pacing and shocking, the connector further includes a right ventricular tip terminal (V_{R} TIP) 52, a right ventricular ring terminal (V_{R} RING) 54, a right ventricular shocking terminal (RV COIL) 56, and an SVC shocking terminal (SVC COIL) 58, which are adapted for connection to the right ventricular tip electrode 32, the right ventricular ring electrode 34, the right ventricular coil electrode 36, and the SVC coil electrode 38, respectively.

At the core of the stimulation device 10 is a programmable microcontroller 60, which controls the various modes of stimulation therapy. As is well known in the art, the microcontroller 60 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 60 includes the ability to process or monitor input signals (data) as controlled by a program code stored in a designated block of memory. The details of the design and operation of the microcontroller 60 are not critical to the invention. Rather, any suitable microcontroller 60 may be used that carries out the functions described herein. The use of microprocessor-based control circuits for performing timing and data analysis functions are well known in the art.

As shown in FIG. 3, an atrial pulse generator 70 and a ventricular pulse generator (Vtr. Pulse Generator) 72 generate pacing stimulation pulses for delivery by the right atrial lead 20, the right ventricular lead 30, and/or the coronary sinus lead 24 via an electrode configuration switch 74. It is understood that in order to provide stimulation therapy in each of the four chambers of the heart, the atrial and ventricular pulse generators, 70 and 72, may include dedicated, independent pulse generators, multiplexed pulse generators, or shared pulse generators. The pulse generators, 70 and 72, are controlled by the microcontroller 60 via appropriate control signals, 76 and 78, respectively, to trigger or inhibit the stimulation pulses.

The microcontroller 60 further includes a timing control circuit 79 which is used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 74 includes a plurality of switches for connecting the desired electrodes to the appropriate I/O circuits, thereby providing complete electrode programmability. Accordingly, the switch 74, in response to a control signal 80 from the microcontroller 60, determines the polarity of the stimulation pulses (e.g., unipolar, bipolar, combipolar, etc.) by selectively closing the appropriate combination of switches (not shown) as is known in the art.

In one embodiment, the stimulation device 10 may include an atrial sensing circuit (Atr. Sense) 82 and a ventricular sensing circuit (Vtr. Sense) 84. The atrial sensing circuit 82 and ventricular sensing circuit 84 may also be selectively coupled to the right atrial lead 20, coronary sinus lead 24, and the right ventricular lead 30, through the switch 74 for detecting the presence of cardiac activity in each of the four chambers of the heart. Accordingly, the atrial sensing circuit 82 and ventricular sensing circuit 84 may include dedicated sense amplifiers, multiplexed amplifiers, or shared amplifiers. The switch 74 determines the “sensing polarity” of the cardiac signal by selectively closing the appropriate switches, as is also known in the art. In this way, the clinician may program the sensing polarity independent of the stimulation polarity. Each sensing circuit, 82 and 84, may employ one or more low power, precision amplifiers with programmable gain and/or automatic gain control, bandpass filtering, and a threshold detection circuit, as known in the art, to selectively sense the cardiac signal of interest. The bandpass filtering may include a bandpass filter that passes frequencies between 10 and 70 Hertz (Hz) and rejects frequencies below 10 Hz or above 70 Hz. The automatic gain control enables the stimulation device 10 to deal effectively with the difficult problem of sensing the low amplitude signal characteristics of atrial or ventricular fibrillation. The outputs of the atrial and ventricular sensing circuits 82 and 84 are connected to the microcontroller 60 which, in turn, is able to trigger or inhibit the atrial and ventricular pulse generators, 70 and 72, respectively, in a demand fashion in response to the absence or presence of cardiac activity in the appropriate chambers of the heart.

For arrhythmia detection, the stimulation device 10 may utilize the atrial and ventricular sensing circuits 82 and 84 to sense cardiac signals to determine whether a rhythm is physiologic or pathologic. The timing intervals between sensed events (e.g., P-waves, R-waves, and depolarization events associated with fibrillation which are sometimes referred to as “F-waves” or “Fib-waves”) are then classified by the microcontroller 60 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks, collectively referred to as “tiered therapy”). Similar capabilities would exist on the atrial channel with respect to tachycardias occurring in the atrium. These would be atrial tachycardias (AT), more rapid atrial tachycardias (Atrial Flutter) and atrial fibrillation (AF).

In another embodiment, the stimulation device 10 may include an analog-to-digital (A/D) data acquisition circuit 90. The data acquisition circuit 90 is configured to acquire an intracardiac signal, convert the raw analog data of the intracardiac signal into a digital signal, and store the digital signals for later processing and/or telemetric transmission to an external device 102. The data acquisition circuit 90 is coupled to the right atrial lead 20, the coronary sinus lead 24, and the right ventricular lead 30 through the switch 74 to sample cardiac signals across any pair of desired electrodes. As shown in FIG. 3 the microcontroller 60 generates a control signal 92 to control operation of the data acquisition circuit 90.

The microcontroller 60 includes an arrhythmia detector 77, which operates to detect an arrhythmia, such as tachycardia and fibrillation, based on the intracardiac signal. The arrhythmia detector 77 senses R-waves in the intracardiac signal, each of which indicates a depolarization event occurring in the heart 12. The arrhythmia detector 77 may sense an R-wave by comparing a voltage amplitude of the intracardiac signal with a voltage threshold value. If the voltage amplitude of the intracardiac signal exceeds the voltage threshold value, the arrhythmia detector 77 senses the R-wave. The arrhythmia detector 77 may also determine an event time for the R-wave occurring at a peak voltage amplitude of the R-wave. The arrhythmia detector 77 may receive an analog intracardiac signal from the sensing circuits 82 and 84 or a digital intracardiac signal from the data acquisition circuit 90. Alternatively, the arrhythmia detector 77 may use the digitized intracardiac signal stored by the data acquisition circuit 90.

The microcontroller 60 may include a morphology detector 99 for confirming R-waves. The morphology detector 99 compares portions of the intracardiac signal with templates of known R-waves to confirm R-waves sensed in the intracardiac signal. In various embodiments, the morphology detector 99 is optional.

The microcontroller 60 is further coupled to a memory 94 by a suitable computer bus 96 (e.g., an address and data bus), wherein the programmable operating parameters used by the microcontroller 60 are stored and modified, as required, in order to customize the operation of the stimulation device 10 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart 12 within each respective tier of therapy. Other pacing parameters include base rate, rest rate and circadian base rate.

Advantageously, the operating parameters of the stimulation device 10 may be non-invasively programmed into the memory 94 through a telemetry circuit 100 in telemetric communication with the external device 102, such as a programmer, transtelephonic transceiver, or a diagnostic system analyzer. The telemetry circuit 100 is activated by the microcontroller 60 by a control signal 106. The telemetry circuit 100 advantageously allows intracardiac electrograms and status information relating to the operation of the stimulation device 10 (as contained in the microcontroller 60 or memory 94) to be sent to the external device 102 through an established communication link 104.

The stimulation device 10 may further include a physiologic sensor 108, commonly referred to as a “rate-responsive” sensor because it is typically used to adjust pacing stimulation rate according to the exercise state of the patient. However, the physiologic sensor 108 may further be used to detect changes in cardiac output, changes in the physiological condition of the heart, or diurnal changes in activity (e.g., detecting sleep and wake states). Accordingly, the microcontroller 60 responds by adjusting the various pacing parameters (such as rate, AV Delay, V-V Delay, etc.) at which the atrial and ventricular pulse generators, 70 and 72, generate stimulation pulses. (V-V delay is typically used only in connection with independently programmable RV and LV leads for biventricular pacing.) While shown as being included within the stimulation device 10, it is to be understood that the physiologic sensor 108 may also be external to the stimulation device 10, yet still be implanted within or carried by the patient. A common type of rate responsive sensor is an activity sensor, such as an accelerometer or a piezoelectric crystal, which is mounted within the case 40 of the stimulation device 10. Other types of physiologic sensors are also known, for example, sensors that sense the oxygen content of blood, respiration rate and/or minute ventilation, pH of blood, ventricular gradient, etc. However, any sensor may be used which is capable of sensing a physiological parameter that corresponds to the exercise state of the patient.

The stimulation device additionally includes a battery 110, which provides operating power to all of the circuits shown in FIG. 3. For the stimulation device 10, which employs shocking therapy, the battery 110 should be capable of operating at low current drains for long periods of time, and then be capable of providing high-current pulses (for capacitor charging) when the patient requires a shock pulse. The battery 110 should also have a predictable discharge characteristic so that elective replacement time can be detected. Accordingly, the stimulation device 10 may employ lithium/silver vanadium oxide batteries. As further shown in FIG. 3, the stimulation device 10 is shown as having a measuring circuit 112 which is enabled by the microcontroller 60 via a control signal 114.

In the case where the stimulation device 10 is intended to operate as an implantable cardioverter/defibrillator (ICD) device, the stimulation device 10 detects and confirms the occurrence of an arrhythmia, and automatically applies an appropriate antitachycardia pacing therapy or electrical shock therapy to the heart 12 for terminating the detected arrhythmia. To this end, the microcontroller 60 further controls a shocking circuit 116 by way of a control signal 118. The shocking circuit 116 generates shocking pulses of low (up to 0.5 joules), moderate (0.5-10 joules), or high energy (11 to 40 joules), as controlled by the microcontroller 60. Such shocking pulses are applied to the patient's heart 12 through at least two shocking electrodes, and as shown in this embodiment, selected from the left atrial coil electrode 28, the right ventricular coil electrode 36, and/or the SVC coil electrode 38. As noted above, the case 40 may act as an active electrode in combination with the right ventricular coil electrode 36, or as part of a split electrical vector using the SVC coil electrode 38 or the left atrial coil electrode 28 (i.e., using the right ventricular coil electrode as a common electrode).

Cardioversion shocks are of relatively low to moderate energy level (so as to minimize the current drain on the battery) and are usually between 5 to 20 joules. Typically, cardioversion shocks are synchronized with an R-wave. Defibrillation shocks are generally of moderate to high energy level (i.e., corresponding to thresholds in the range of 5 to 40 joules), delivered asynchronously (since R-waves may be too disorganized), and pertaining exclusively to the treatment of fibrillation. Accordingly, the microcontroller 60 is capable of controlling the synchronous or asynchronous delivery of the shocking pulses.

### Feedthrough Header Assembly

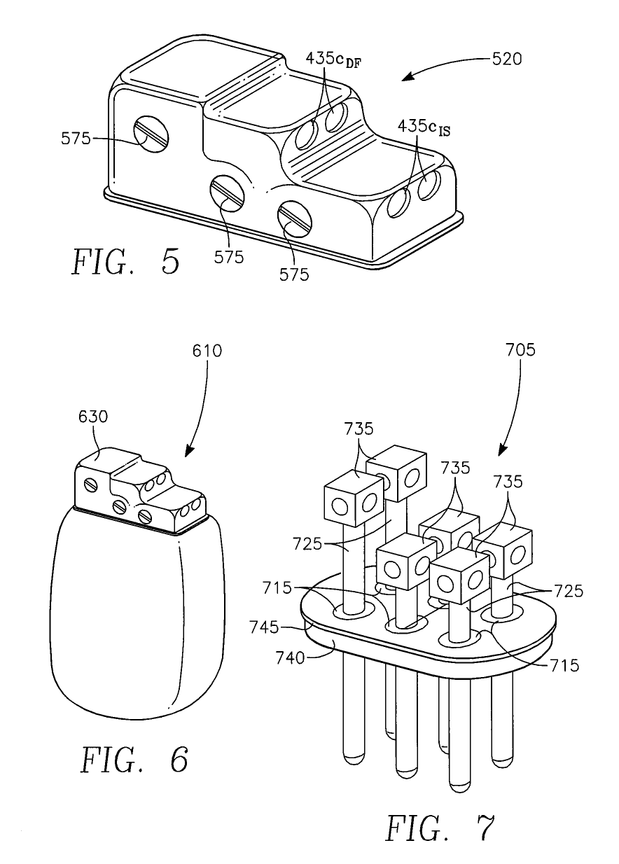

FIG. 4 shows exploded view drawing showing a portion of a feedthrough assembly 405 of the implantable cardiac device 410. The feedthrough posts 425 are straight and extend directly between the connector blocks 435 through the feedthrough dielectric 415. The conductor posts 425 may be titanium posts manufactured to include the connector blocks 435 for electrical contact with lead pins & rings (not shown). Thus, the connector blocks 435 and the conductor posts 425 are integrally formed, for example by machining, or by metal injection molding. The connector blocks 435 may include holes 435p and 435r to accept the pins and rings (not shown), respectively, as well as holes 435s for setscrews 575 (shown in FIG. 5), to establish electrical contact and accomplish mechanical retention of leads (not shown).

Titanium conductor posts 425 may be brazed to a ceramic feedthrough dielectric 415. The feedthrough header assembly 405 may also contain discoidal capacitors (not shown), which will connect to the posts to form an EMI filter. Typically there is one filter per conductor post 425. The capacitors (not shown) may be embedded within the feedthrough dielectric 415.

A titanium collar 440 with flange 445 encloses the feedthrough dielectric 415 and conductor posts 425. The flange 445 may be welded to the device case 465, which also may be made of titanium.

FIG. 5 shows a perspective view of a header 520. Referring to FIGS. 4 and 5, the header 520 covers the feedthrough assembly 405. The header 520 may be molded/cast directly on the feedthrough assembly 405, with cavities 435c_{DF} and 435c_{IS} formed to allow connection of leads (not shown) to the connector blocks 435. The connector blocks 435 on the conductor posts 425 will form the IS-1 and DF-1 connector cavities 435c_{DF} and 435c_{IS}, respectively. Although a six pole device is shown for illustration purposes, other embodiments may have a different number of connector blocks and conductor posts.

FIG. 6 shows a perspective view of an implantable cardiac device 610 with an attached header assembly 630.

Referring to FIG. 4, the posts 425 of the feedthrough assembly 405 may extend through the dielectric feedthrough to allow electrical connection of the feedthrough posts 425 with a printed wire board (not shown) within the case 465, for example via a flex cable (not shown), or other know connection means.

The case 465 and feedthrough 405 are typically hermetically sealed and also provide shielding from electromagnetic noise or other interference signals. The feedthrough 405 is the interface between the leads (not shown) and the electronics (not shown) inside the case 465.

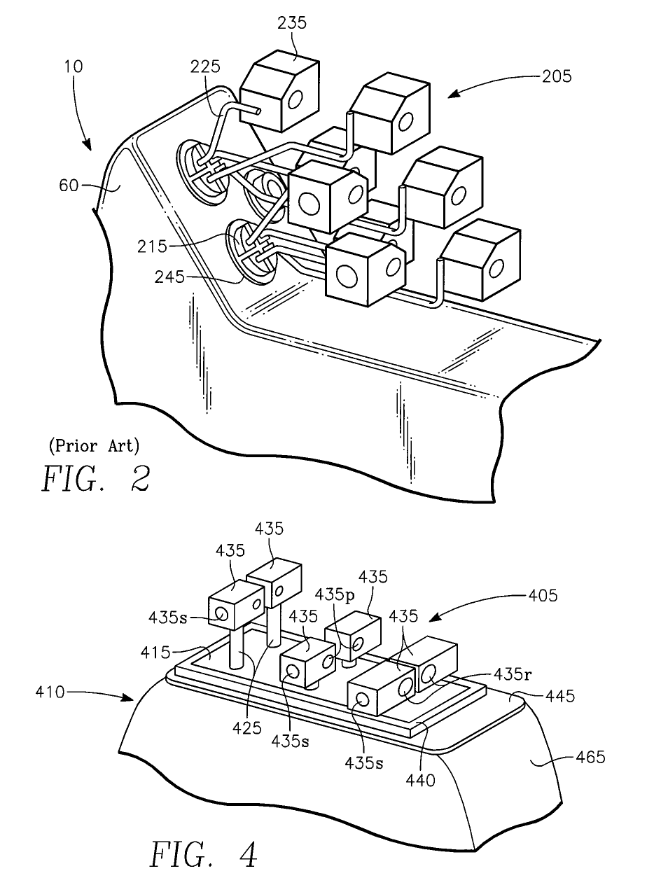

With the conventional implantable cardiac device of FIG. 2, the feedthrough dielectrics 215 are closely spaced on an small inclined edge portion of the case 60 between a top and side edges of the case 60. In the embodiment of FIG. 4, the feedthrough dielectric is a significant portion, of the upper edge of the case 465.

One benefit of having more spacing between posts 425 in the feedthrough 405 is that because there can be over 800V sent through the posts when shocking, it is helpful to space the posts 425 farther apart in the dielectric 415 to inhibit shorting and break down of the dielectric.

With conventional configurations, such as shown in FIG. 2, the platinum iridium wires have to be formed so that the blocks are in the proper location. With the embodiment of FIG. 4, however, the posts are spaced within the dielectric 415 so that the connector blocks are positioned exactly where they need to be, rather than bending the platinum indium wires 225 to position the connector blocks 435.

In various embodiments, the conductor posts provide a wireless feedthrough for a header assembly. In some embodiments, the integral posts need not have a bigger diameter than the conventional wires, and may be curved in some embodiments. As disclosed above, in some embodiments, the feedthrough flange can be welded/braised to the case, so no backfilling is required.

FIG. 7 shows a possible embodiment of the feedthrough assembly 705 having individual dielectric rings 715 for each of the conductor posts 725. The dielectric rings 715 may be ceramic and further include a capacitor embedded in each of the rings 715, i.e. a capacitor associated with each of the conductor posts 725. In this embodiment, the collar 740 and flange 745 encircle each of the conductor posts 725 individually, rather than collectively as in the embodiment shown in FIG. 4.

Various embodiments of the present invention allow reduced manufacturing complexity by eliminating wire forming and wire to block welding. Moreover, various embodiments may provide greater system reliability, with the possibility of shorting between wire and case virtually eliminated. Further, in various embodiments, the resistance may be reduced by decreased conductor post length and increased cross-sectional area of posts. In addition, various embodiments allow use of conductor material other than platinum iridium to significantly reduce header cost.

Although exemplary methods, devices, systems, etc., have been described in language specific to structural features and/or methodological acts, it is to be understood that the subject matter defined in the appended claims is not necessarily limited to the specific features or acts described. Rather, the specific features and acts are disclosed as exemplary forms of implementing the claimed methods, devices, systems, etc.

What is claimed is:

1. An implantable cardiac device comprising:
a) a case;
b) a feedthrough comprising a dielectric extending through the case;
c) a feedthrough conductor comprising a connector block and a conductor post integrally formed with the connector block, the conductor post extending from the connector block through the dielectric; and
d) a capacitor within the dielectric.

2. The device of claim 1, wherein the conductor post is a straight conductor post extending from a side of the connector block facing the feedthrough directly toward the feedthrough.

3. The device of claim 2, wherein the conductor post and the connector block are formed of the same material.

4. The device of claim 3, wherein the conductor post and the connector block comprise one of titanium, MP35N, stainless steel, palladium, or platinum-iridium.

5. The device of claim 1, comprising a plurality of straight conductor posts and a plurality of connector blocks, each of the plurality of conductor posts being integrally formed with a respective connector block, and wherein the dielectric comprises a single matrix dielectric, each of the plurality conductor posts extending from the connector block through the single matrix dielectric.

6. The device of claim 1, comprising a plurality of conductor posts and a plurality of connector blocks, each of the plurality of conductor posts being integrally formed with a respective connector block, and wherein the feedthrough comprises a plurality of dielectric portions, each of the plurality conductor posts extending through the case through a separate dielectric portion.

7. The device of claim 6, wherein the dielectric portions comprises ceramic rings.

8. The device of claim 1, wherein the conductor post and the connector block are formed of the same material.

9. The device of claim 8, wherein the conductor post and the connector block comprise one of titanium, MP35N, stainless steel, palladium, or platinum-iridium.

10. The device of claim 1, wherein the dielectric comprises ceramic.

11. The device of claim 1, wherein the feedthrough comprises a flange surrounding the dielectric, the flange being secured to the case.

12. An implantable cardiac device comprising:
a) a case;
b) a feedthrough comprising a single matrix ceramic;
c) a plurality of feedthrough conductors each comprising a connector block and a straight conductor post integrally formed with the connector block and extending from the connector block through the dielectric single matrix ceramic; and
d) at least one capacitor within the single matrix ceramic.

13. The device of claim 12, wherein the straight conductor post and the connector block are formed of the same material.

14. The device of claim 13, wherein the straight conductor post and the connector block comprise one of titanium, MP35N, stainless steel, palladium, or platinum-iridium.

15. The device of claim 12, wherein the feedthrough comprises a flange surrounding the dielectric, the flange being secured to the case.

16. The device of claim 12, further comprising a plurality of capacitors within the single matrix ceramic, each being connected to a corresponding straight conductor post.

17. An implantable cardiac device comprising:
a) a case;
b) a feedthrough comprising a plurality of ceramic rings;
c) a plurality of feedthrough conductors each comprising a connector block and a straight conductor post integrally formed with the connector block and extending from the connector block through a respective one of the plurality of ceramic rings; and
d) a capacitor within each of the plurality of ceramic rings.

18. The device of claim 17, wherein the conductor post and the connector block are formed of the same material.

19. The device of claim 18, wherein the conductor post and the connector block comprise one of titanium, MP35N, stainless steel, palladium, or platinum-iridium.

20. The device of claim 17, wherein the feedthrough comprises a flange surrounding the plurality of ceramic rings, the flange being secured to the case.
